(19) United States
(12) Patent Application Publication
(10) Pub. No.: US 2019/0284625 A1
(43) Pub. Date: Sep. 19, 2019

(54) METHODS FOR JOINT LOW-PASS AND TARGETED SEQUENCING

(71) Applicant: Gencove Inc., New York, NY (US)

(72) Inventors: Joseph Pickrell, Brooklyn, NY (US); Tomaz Berisa, Brooklyn, NY (US); Kaja Wasik, Bethpage, NY (US)

(21) Appl. No.: 16/354,575

(22) Filed: Mar. 15, 2019

Related U.S. Application Data
(60) Provisional application No. 62/644,183, filed on Mar. 16, 2018.

(51) Int. Cl. C12Q 1/6874

(52) U.S. Cl. CPC C12Q 1/6874

(57) ABSTRACT

The present disclosure provides a method for analyzing a genetic sample comprising dividing a library into at least two subsets, enriching one of the at least two subsets, and pooling the enriched and unenriched subsets before sequencing the sample. The present disclosure also provides an enriched genomic library comprising both a target-enriched subset and an unenriched subset of the library.

### RELATED APPLICATIONS

This application claims the benefit of priority to U.S. Provisional Patent Application having Ser. No. 62/644,183, filed Mar. 16, 2018, the content of which is hereby incorporated herein by reference in its entirety.

### BACKGROUND

A major goal of human genetics is to identify the genetic variants that influence diseases and other traits. It has become clear that for many traits this requires extremely large sample sizes, at least in the hundreds of thousands of individuals. Currently, the technology of choice for large-scale genomics work is the genotyping array. An alternative, low-pass sequencing, increases power and allows for the discovery of new genetic variants. One key limitation of low-pass sequencing is that there is a stochastic aspect to which genetic variants are well-measured. Provided herein is an approach to combine the increased genome-wide power of low-pass sequencing with the programmable quality of genotyping arrays using capture technologies.

### SUMMARY OF THE INVENTION

In certain aspects, the present disclosure provides methods for analyzing a genetic sample comprising dividing a genetic library into a first subset and a second subset; and enriching the first subset of the genetic library for a set of one or more target genetic loci or regions, thereby creating a target-enriched subset. The genetic library may be barcoded and consist of multiple samples.

In another aspect, provided herein is an enriched genomic library comprising a target-enriched subset of a genetic library and an unenriched subset of a genetic library. The genetic library may be barcoded and consist of multiple samples.

### BRIEF DESCRIPTION OF THE DRAWINGS

FIG. 1 shows a schematic of the library preparation steps of the method. The lines represent DNA molecules, the circle represents a genetic locus or region, and the rectangles represent indices that uniquely tag each input sample. After step 5, the enriched library is sequenced and then computationally de-multiplexed.

FIG. 2A shows a graph of the average coverage from a set of 32 pooled libraries.

FIG. 2B shows a graph of the minimum coverage from a set of 32 pooled libraries.

FIG. 3A shows a graph of the average coverage from a set of 48 pooled libraries.

FIG. 3B shows a graph of the minimum coverage from a set of 48 pooled libraries.

### DETAILED DESCRIPTION OF THE INVENTION

In certain aspects, the present disclosure provides a method for targeted sequencing, comprising: dividing a genetic library into a first subset and a second subset; and enriching the first subset of the genetic library for a set of one or more target genetic loci or regions, thereby creating a target-enriched subset. In further embodiments, the method further comprises adding the target-enriched subset of the genetic library to the second subset of the genetic library to generate a target-enriched sequencing library pool.

In certain embodiments, the genetic library is barcoded. In some embodiments, the genetic library comprises genomic DNA.

In certain embodiments, the genetic library comprises DNA from a tissue.

In certain embodiments, the genetic library comprises DNA from a sample. In certain embodiments, the genetic library comprises DNA from a plurality of samples. In certain embodiments, the sample or samples are obtained from a cheek swab. In certain embodiments, the sample or samples are obtained from saliva. In certain embodiments, the sample or samples are obtained from blood.

In certain embodiments, the genetic library comprises DNA from an individual. In certain embodiments, the genetic library comprises DNA from a population of individuals. In certain embodiments, the individual or individuals are humans. In certain embodiments, the individual or individuals are not humans.

In certain embodiments, a plurality of target-enriched sequencing library pools are prepared and combined into a single pool.

In certain embodiments, the enriching step comprises contacting the genetic library with sequence-specific oligonucleotide probes. In certain embodiment, the oligonucleotide probes are in solution. In certain embodiments, the oligonucleotide probes are immobilized on a surface. In certain embodiments, the oligonucleotide probes are specific for one or more target genomic loci or regions. In certain embodiments, the oligonucleotide probes are specific for known genetic variants.

In certain embodiments, the method further comprises sequencing the target-enriched sequencing library pool thereby generating sequencing reads. In certain embodiments, the sequencing step comprises using a short-read technology. In certain embodiments, the sequencing step comprises using a long-read technology.

In certain embodiments, the sequencing step comprises using low-coverage sequencing. In certain embodiments, low-coverage sequencing comprises providing 10 fold coverage or less of a target genome.

In certain embodiments, the sequencing reads are demultiplexed. The demultiplexed sequencing reads are aligned to a reference genome (e.g., a human reference genome). In certain embodiments, the reference genome is a non-human reference genome.

In certain embodiments, the genetic library is prepared at low-volume.

In certain aspects, the present disclosure provides enriched genetic libraries comprising a target-enriched subset of a genetic library and an unenriched subset of a genetic library. In certain embodiments, the target-enriched subset and the unenriched subset are separate. In certain embodiments the target-enriched subset and the unenriched subset are pooled. In certain embodiments, the target-enriched subset is specific for genomic loci or regions. In certain embodiments, the target-enriched subset is specific for one or more genetic variants. In certain embodiments, the genetic library comprises genomic DNA.

### Biological Samples

Genetic samples may be procured from more than one individual. Genetic samples may be procured from a plurality of individuals, for example several hundred, several thousand, or a million or more individuals.

As used herein, “genetic sample” means any sample of material comprising genetic information, for example DNA (including genomic, mitochondrial, chloroplast, plasmid and eDNA) or RNA (including processed or unprocessed mRNA, tRNA, rRNA and miRNA). In one embodiment, the genetic material comprises DNA. In another embodiment, the genetic material comprises genomic DNA.

In certain embodiments, the genetic library sample comprises genomic DNA. As used herein “deoxyribonucleic acid” (DNA) is a, usually double-stranded, long molecule that is used by biological cells to encode other shorter molecules, such as proteins, used to build and control all living organisms. DNA is composed of repeating chemical units known as “nucleotides” or “bases.” There are four bases: adenine, thymine, cytosine, and guanine, represented by the letters A, T, C and G, respectively. Adenine on one strand of DNA always binds to thymine on the other strand of DNA; and guanine on one strand always binds to cytosine on the other strand and such bonds are called base pairs. Any order of A, T, C and G is allowed on one strand, and that order determines the reverse complementary order on the other strand. The actual order determines the function of that portion of the DNA molecule. Information on a portion of one strand of DNA can be captured by ribonucleic acid (RNA) that also is composed of a chain of nucleotides in which uracil (U) replaces thymine (T). Determining the order, or sequence, of bases on one strand of DNA or RNA is called sequencing. A portion of length k bases of a strand is called a k-mer; and specific short k-mers are called oligonucleotides or oligomers or “oligos” for short. The base found at one location (locus) on the strand is called the value at that locus.

In other embodiments, the genetic library sample may comprise DNA from a tissue, individual, or population of individuals. In preferred embodiments, the barcode on the genetic sample corresponds to the origin of the genetic material.

In other embodiments, the first subset of the library may be enriched for a specific target by contacting the first subset of the library with a sequence-specific oligonucleotide probe immobilized on a surface. The oligonucleotide probe may be in solution. The oligonucleotide probe may be specific for a genomic locus, region, or a known genetic variant.

### Probes

As one of skill in the art appreciates, the probes described herein can take on a variety of configurations and may have a variety of structural components. For example, a “locus specific” probe may be a probe that hybridizes to a target sequence in a locus specific manner, but does not necessarily discriminate between alleles. The size of the oligonucleotide probe may vary, as will be appreciated by those in the art, with each portion of the probe and the total length of the probe in general varying from 5 to 500 nucleotides in length. A locus specific probe or probes may comprise a target domain substantially complementary to the target sequence, such that hybridization of the target and the probes occurs.

Probes may further comprise adapter sequences, sometime referred to in the art as “zip codes” or “bar codes.” Adapters facilitate immobilization of probes to allow the use of “universal arrays.” That is, arrays (either solid phase or liquid phase arrays) are generated that contain capture probes that are not target specific, but rather specific to individual (preferably) artificial adapter sequences. Thus, an “adapter sequence” is a nucleic acid that is generally not native to the target sequence, i.e. is exogenous, but is added or attached to the target sequence. The terms “barcodes”, “adapters”, “addresses”, “tags” and “zipcodes” have all been used to describe artificial sequences that are added to genetic samples to allow separation of nucleic acid fragment pools. Adapters serve as unique identifiers of the probe and thus of the target sequence.

As will be appreciated by those in the art, the attachment, or joining, of the adapter sequence to the target sequence can be done in a variety of ways (e.g., enzymatically). The adapter may be attached either on the 3′ or 5′ ends.

In certain embodiments, the first and second subsets of the library are combined to generate a target-enriched sequencing library pool. In certain embodiments, the target-enriched sequencing library pool may comprise any suitable ratio of enriched genetic material to unenriched genetic material, for example, about 100:1, about 90:1, about 80:1, about 70:1, about 60:1, about 50:1, about 40:1, about 30:1, about 20:1, about 10:1, about 8:1, about 6:1, about 4:1, about 2:1, about 1:1, about 1:2, about 1:4, about 1:6, about 1:8, about 1:10, about 1:20, about 1:30, about 1:40, about 1:50, about 1:60, about 1:70, about 1:80, about 1:90, or about 1:100. In certain embodiments, the ratio of enriched genetic material to unenriched genetic material is from about 100:1 to about 1:1, from about 30:1 to about 1:1, from about 10:1 to about 1:1, or from about 3:1 to about 1:1. In certain embodiments, the ratio of enriched genetic material to unenriched genetic material is from about 1:1 to about 1:100, from about 1:1 to about 1:30, from about 1:1 to about 1:10, or from about 1:1 to about 1:3.

### Sequencing

In other embodiments, the target-enriched sequencing library pool is sequenced thereby generating sequencing reads. The target-enriched sequence library may be sequenced using short-read technology or long-read technology. In a preferred embodiment, the target-enriched sequence library is sequenced using low-coverage sequencing. Low-coverage sequencing may be 10× (or 10-fold) coverage or less of a target genome, for example about 9×, 8×, 7×, 6×, 5×, 4×, 3×, 2×, or 1× coverage of the target genome. Compositions and methods related to low-coverage sequences are described, for example, in U.S. Patent Application Publication No. 2018/004730 by Pickrell et al, the contents of which are fully incorporated by reference herein. In an embodiment, the sequencing reads are demultiplexed and aligned to one or more reference genome. In a preferred embodiment, the reference genome comprises a human reference genome.

As used herein, “low-coverage sequencing” refers to the amount of coverage obtained by sequencing with respect to a set of reference genetic material, such as the genome of an organism. For example, only a fraction of the reference genetic material may be represented by the sequenced material from the genetic sample; e.g., about 10× coverage or less of the reference genetic material. In some embodiments, low coverage sequencing means less than 10× coverage of the reference genetic material, for example about 9×, 8×, 7×, 6×, 5×, 4×, 3×, 2×, 1×, 0.5×, 0.4×, 0.3×, 0.2×, or 0.1× coverage of the reference genetic material. As used herein, low-coverage sequencing can also refer to range of coverage of the reference genetic material, for example between about 0.1× to about 10×, about 0.8× to about 8×, about 0.1× to about 5× and about 0.4× to about 4×.

One of ordinary skill in the art can readily determine the sequencing coverage of reference genetic material obtained when sequencing a genetic sample according to the present methods. For example, the number of sequencing reads covering the known polymorphic sites in the reference genomes across the genetic samples being tested can be counted, and the coverage determined by comparing the variation in the number of sequencing reads.

Any suitable technique for sequencing genetic material from the one or more genetic samples may be used in various embodiments of the present methods. Apparatuses and materials for carrying out such sequencing techniques are well-known in the art, and are commercially available. For example, suitable sequencing machines and protocols are available from Illumina, Inc. of San Diego, Calif. as the Illumina MiSeq or Illumina HiSeq 2500. The sequencing results can be in any standard output format that is suitable for storage and retrieval in a database, and/or for further analysis, as are well-known to one of ordinary skill in the art; for example, in in FASTQ format. In some embodiments, the output is demultiplexed, for example so that a single FASTQ file corresponds to a single identified (e.g., barcoded) sample.

### Sample Collection

Biological samples may be procured in any manner suitable for subsequent isolation of genetic material, for example by collecting or drawing a bodily fluid such as blood, lymph, sweat, saliva, urine, tears, synovial fluid, cerebrospinal fluid, and the like. The sample may be collected into any suitable container.

Blood may be collected into a vacuum tube (e.g., Vacutainer, Becton, Dickinson & Co., Franklin Lakes, N.J.), test tube or capillary tube. The blood may be separated into its component parts prior to isolation of genetic material. If the blood is separated into its component parts, genetic material is isolated from the fraction containing nucleated cells (e.g., white blood cells or hematopoietic stem cells). In some embodiments, any collected whole or fractionated blood is stored for later extraction of genetic material, for example under conditions (such as refrigeration or in a stabilizing solution) which would preserve the integrity of the genetic material such that, upon extraction, it could be subject to the methods of the various embodiments. Collected whole or fractionated blood may be packaged and shipped to a facility for subsequent extraction of genetic material. Suitable blood collection techniques, blood collection and storage containers, and blood storage and shipping techniques used in various embodiments, are well-known to those of ordinary skill in the art.

Saliva may be collected by any number of suitable techniques well-known to those of ordinary skill in the art, and include, for example, the SS-SAL-1 or SS-SAL-2 saliva DNA collection devices available from SpectrumDNA (Draper, Utah). Saliva may be procured from an individual by having the individual spit into the collection device, which, may contain a solution which stabilizes the saliva sample, and inhibits bacterial growth. The saliva collection device may be packaged and shipped to a facility for subsequent extraction of genetic material from the individual's cells and/or from organisms (such as bacteria) contained within the saliva sample. Other suitable saliva collection techniques, saliva collection and storage containers, and saliva storage and shipping techniques used in various embodiments, are well-known to those of ordinary skill in the art.

Other suitable biological samples for use in the present methods comprise cells or tissue from an individual that are not necessarily derived from bodily fluids. For example, in some embodiments, suitable biological samples comprise epithelial cells, such as those obtained by a swab of bodily surfaces such as the inside of the mouth, nasal passages, vaginal or rectal surfaces, or the skin. In some embodiments, suitable biological samples comprise tissue or non-epithelial cells, such as obtained by a biopsy or by isolating and culturing cells from the individual. Techniques for obtaining, shipping storing and/or culturing tissue or cellular samples from an individual used in various embodiments, are well-known to those of ordinary skill in the art.

In certain embodiments, the genetic sample may be obtained from a cheek swab, saliva, or blood of a human. In preferred embodiments, the genetic sample is obtained from a cheek swab.

Any suitable technique for extracting genetic material from an individual's biological sample may be used. Such techniques typically employ mechanical, enzymatic and/or chemical means to lyse the cells comprising the biological sample, to free the nucleus and cytoplasm, and then either the nucleus or cytoplasm is subjected to a number of isolation and fractionation steps designed to sequentially and substantially separate the genetic material from the non-genetic material (e.g., cellular debris and other components) of the biological samples. Such techniques also typically employ one or more steps or substances which preserve the integrity of any genetic material e.g., DNA or RNA), for example by inactivating any nucleases which may be present in the biologic sample.

### Genetic Library Preparation

The samples described above may be used to generate a genetic library comprising sequenceable material. Any suitable technique known to one of ordinary skill in the art, including the fragmentation, tagging of genetic material with sequencing adaptors to provide sequenceable material may be used to generate sequenceable material. Suitable library preparation techniques are described in, for example, Picelli S et al. (2016), Tn5 transposase and tagmentation procedures for massively scaled sequencing projects, Genome Research 24:2033-2040; Baym Metal. (2015), Inexpensive multiplexed library preparation for megabase-sized genomes, PLosOne 10(5): e0128036 (DOI:10.1371/journal.pone.0128036; and Adey A et al. (2010), Rapid, low-input, low-bias construction of shotgun fragment libraries by high-density in vitro transposition, Genome Biology 11:R119, the entire disclosures of which are herein incorporated by reference. Suitable materials and protocols for library preparation are also commercially available, such as the Nextera XT DNA library prep Kit from Illumina, Inc. (San Diego, Calif.), which can be used according to the manufacturer's protocol, and which combines the steps of DNA fragmentation, end-polishing, and adaptor-ligation into one step called “tagmentation” (see, e.g., Picelli S et al. (2016), supra).

In certain embodiments, the library may be prepared at low-volume. As used herein, a “low-volume” reaction means that the total reaction volume is less than that of the standard reaction. In some embodiments, a low-volume reaction can be about ½, ⅓, ¼, ⅕, ⅙, 1/7, ⅛, 1/9, 1/10, 1/12, 1/15, 1/20, 1/25 or 1/30 of the standard reaction volume. In the context of library preparation used in the present methods, a low-volume reaction can be about 50 μl or less, such as 45 μl, 40 μl, 35 μl, 30 μl, 25 μl, 22.5 μl, 20 μl, 15 μl, 10 μl, 5 μl, 1 μl, 0.5 μl or less than 0.5 μl. The low-volume reaction may allow for more reactions to be performed more quickly, and at a reduced cost. Genetic libraries made according to the present methods can be further analyzed prior to sequencing, for example by determining the nucleic acid size concentration or size distributions.

In another aspect, provided herein is an enriched genetic library comprising a pool of enriched and unenriched genetic material. In an embodiment, the enriched genetic material may be specific for one or more genetic variants. The genetic material may be specific for a genomic locus or region. The genetic material may be genomic DNA. In certain embodiments, the library may comprise any suitable ratio of enriched genetic material to unenriched genetic material, for example, about 100:1, about 90:1, about 80:1, about 70:1, about 60:1, about 50:1, about 40:1, about 30:1, about 20:1, about 10:1, about 8:1, about 6:1, about 4:1, about 2:1, about 1:1, about 1:2, about 1:4, about 1:6, about 1:8, about 1:10, about 1:20, about 1:30, about 1:40, about 1:50, about 1:60, about 1:70, about 1:80, about 1:90, or about 1:100. In certain embodiments, the ratio of enriched genetic material to unenriched genetic material is from about 100:1 to about 1:1, from about 30:1 to about 1:1, from about 10:1 to about 1:1, or from about 3:1 to about 1:1. In certain embodiments, the ratio of enriched genetic material to unenriched genetic material is from about 1:1 to about 1:100, from about 1:1 to about 1:30, from about 1:1 to about 1:10, or from about 1:1 to about 1:3.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about ×” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### EXAMPLES

The invention now being generally described, it will be more readily understood by reference to the following examples which are included merely for purposes of illustration of certain aspects and embodiments of the present invention, and are not intended to limit the invention.

### Example 1: Experimental Design

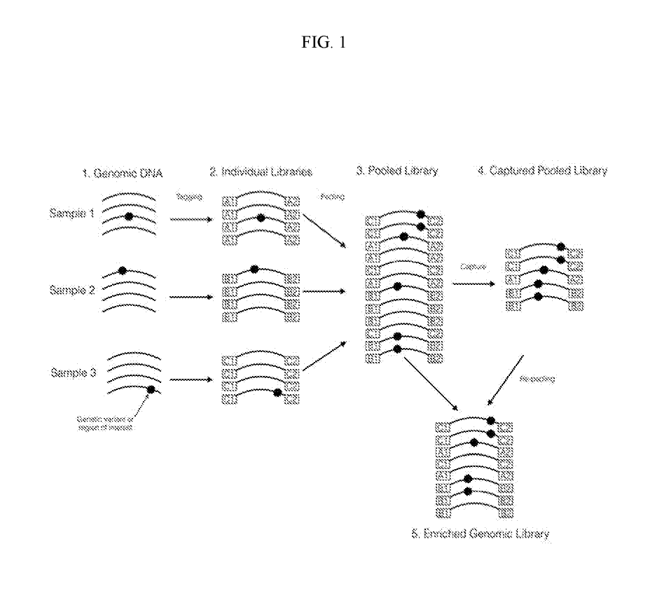

A fragmentation and tagging assay was performed on a set of DNA samples (in practice 48, 96, or 384, though in principle there is no upper limit to the number that can be prepared at once) and the fragmented and tagged DNA was amplified with a set of barcoded primers (FIG. 1). For this any commercial or custom sequencing library preparation system can be used (i.e. Roche, Illumina, Neb., etc.). The individual, barcoded libraries were then pooled and a portion of this pool was saved for a low-pass sequencing assay. In practice pools range from 2 to 384 samples, but in principle the pools can be much larger and encompass thousands of individually barcoded libraries. A targeted DNA enrichment assay was performed on the remainder of the pooled libraries by capturing DNA fragments of interest using hybridization. The pooled capture library could be sequenced on its own or spiked into the not-enriched, sequencing pool, for low coverage sequencing, creating a target enriched sequencing library pool. The target enriched library pool was sequenced and the resulting reads were demultiplexed. In practice, any commercial (or custom) short- or long-read technology (for example, the Illumina sequencing platform) could be used. This provided a random coverage of the input genomes from the low-pass sequencing library pool along with high coverage of the targeted sites from the captured library pool. After demultiplexing, in addition to standard low-pass downstream analysis on the resulting sequencing reads, genotypes for the target capture sites were called.

### Example 2: Low-Pass Sequencing Combined with High Coverage of Specific Genetic Variants

### Preparation of the Genetic Library

DNA, extracted from blood, was obtained from 48 individuals. 81 sequencing libraries were prepared from these DNA samples, varying the amount of input DNA and the amount of reagents used. All libraries were prepared using Kapa Hyper Plus library preparation kit (Roche, cat. #07962428001). The manufacturer's protocol was followed for all the library preparation steps, but the protocol was miniaturized. The modifications of the manufacturer's protocol involved the amount of DNA input, the amount of reagents used, and the number of PCR cycles. The DNA inputs for 81 libraries were as follows: in library 1, 500 ng were used; in libraries 2-17, 200 ng were used; in libraries 18-57, 100 ng were used; and in libraries 58-81, 50 ng were used. The DNA was fragmented for 11 min and 30 seconds. The miniaturization factor used for all libraries were as follows: for library 1, no miniaturization; for libraries: 2-33, one half of recommended volume of all the reagents was used; for libraries 34-81, one fourth of the recommended volume of all the reagents was used. The number of PCR cycles used in each reaction was as follows: for library 1, 2 PCR cycles were used; for libraries 2-33, 6 PCR cycles were used; for libraries 34-81, 7 PCR cycles were used.

### Pooling

Once prepared, all libraries were purified using SPRIselect magnetic beads (cat. # B23318, Beckman Coulter) in a 0.7× ratio of beads to library according to manufacturer's instructions. DNA concentration was measured using Quant-iT PicoGreen Assay (Thermofisher Scientific, cat. # P7589) according to manufacturer's instructions on SpectraMax iD5 (Molecular Devices). The libraries were pooled in equimolar ratios and size selection/concentration was performed using SPRIselect magnetic beads (cat. # B23318, Beckman Coulter) in a 0.7× (left size) and 0.56 (right size) ratio of beads to library according to manufacturer's instructions. The first pool of libraries, for low-pass sequencing, included all 81 libraries and was eluted in 20 of elution buffer (EB) (VWR, Omega-Biotek, PD089). For targeted capture, library 1 was size selected and concentrated on its own and libraries 2-33 and 34-81 were pooled in two separate pools. The three libraries were eluted in 20 μL of EB (VWR, Omega-Biotek, PD089). The DNA concentration of all libraries/pools was measured using Qubit dsDNA High Sensitivity Kit (cat. # Q32854, ThermoFisher Scientific) on a Qubit Fluorometer (ThermoFisher Scientific). Three sequencing libraries from the set of 48 pooled libraries had low concentration and so were excluded from further analysis.

### Capture

In order to perform a proof of concept target capture the xGen® Human ID Research Panel v1.0 (IDT) was tested. The panel is designed to capture 76 distinct, highly polymorphic sites with 229 individually synthesized xGen Lockdown® Probes. The capture was performed on 500 ng of library 1, 3 μg of pooled libraries 2-33, and 4 μg of pooled libraries 34-81. The capture was performed according to manufacturer's description. The final libraries were eluted in 20 μL of EB (VWR, Omega-Biotek, PD089). The DNA concentration was measured using Qubit dsDNA High Sensitivity Kit (cat. # Q32854, ThermoFisher Scientific) on a Qubit Fluorometer (ThermoFisher Scientific). To determine the library size, 1 μL of each library pool was run on Bioanalyzer (Agilent) using the High Sensitivity DNA Analysis Kit (Agilent, cat. #5067-4626).

### Re-Pooling and Sequencing

All the libraries were pooled into a final sequencing pool in the following ratios: 70% of the pool included 81 low-pass sequencing libraries, 10% of the pool comprised library 1 post-target capture, 10% of the pool comprised libraries 2-33 post-target capture, and 10% of the pool comprised libraries 34-81 post-target capture. The libraries were then sequenced using the Illumina HiSeq X Ten system (2×150 bp).

### Analysis

The de-multiplexed sequencing reads were aligned to the human genome reference using bwa mem version 0.7.15-r1140, and PCR duplicates were removed. To assess the coverage of each of the targeted genetic variants, the mpileup command in SAMtools version 1.3.1 was used. Genotypes for each targeted site were called using bcftools version 1.6. Analysis was conducted on the 71 autosomal sites that were targeted.

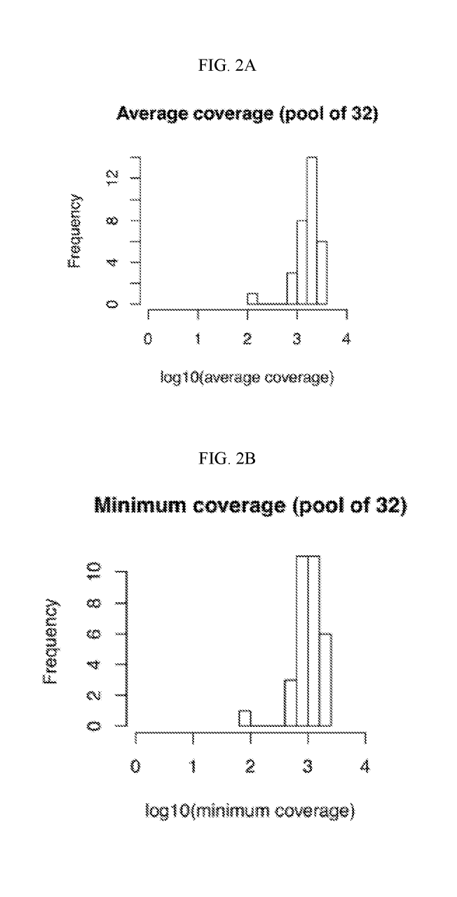

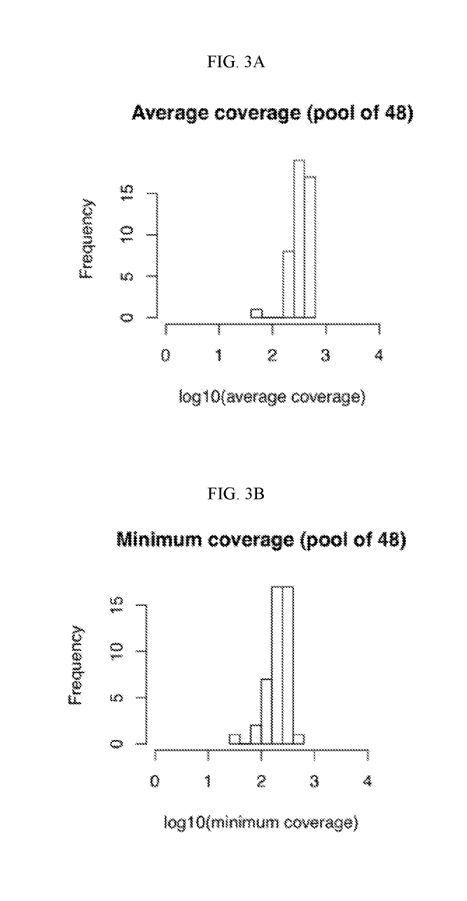

In all 78 libraries (the set of 81 libraries excluding the three where library preparation failed), all 71 autosomal, targeted sites were observed. For simplicity, 5 non-autosomal loci were excluded from the subsequent analysis. In the sample that was not multiplexed (library 1), the average coverage of each site was 3405 sequencing reads, with a minimum coverage across sites of 2248 and a maximum across sites of 4121. The average and minimum coverages for the set of 32 pooled libraries are shown in FIG. 2; the overall average coverage across the 71 autosomal sites was 1769 sequencing reads. For the set of 48 pooled libraries, the average and minimum coverages are shown in FIG. 3; the overall average coverage across the 71 autosomal sites was 356 sequencing reads.

To assess genotype calls, the one sample sequenced three times (once without pooling, once in the pool of 32 samples, and once in the pool of 48 samples) was used. Genotypes from the sequencing reads in each of the three libraries were called using bcftools. Genotypes at all sites were 100% concordant across all three sequencing libraries.

### Example 3: Low-Pass Sequencing Combined with High Coverage of Genomic Regions

DNA from a set of samples is isolated from any source and libraries prepared as in Example 2 (low-pass sequencing and targeted capture). Instead of performing capture for a set of known genetic variants as in Example 2, oligonucleotide probes are designed to capture both a set of genetic loci (e.g., known variants) and a set of genomic regions (e.g., entire exons of a set of genes, introns, or other contiguous regions). The number of samples used for multiplexed capture varies depending on the number of capture targets, desired depth of sequencing coverage, and sequencing method and instrument used.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for targeted sequencing, comprising:
dividing a genetic library into a first subset and a second subset; and
enriching the first subset of the genetic library for a set of one or more target genetic loci or regions, thereby creating a target-enriched subset.

2. The method of claim 1, further comprising adding the target-enriched subset of the genetic library to the second subset of the genetic library to generate a target-enriched sequencing library pool.

3. The method of claim 1, wherein the genetic library is barcoded.

4. The method of claim 1, wherein the genetic library comprises genomic DNA.

5-10. (canceled)

11. The method of claim 1, wherein the genetic library comprises DNA from an individual.

12. The method of claim 1, wherein the genetic library comprises DNA from a population of individuals.

13-14. (canceled)

15. The method of claim 1, comprising preparing a plurality of target-enriched sequencing library pools; and combining the plurality of target-enriched sequencing library pools into a single pool.

16. The method of claim 1, wherein the enriching step comprises contacting the genetic library with sequence-specific oligonucleotide probes.

17. The method of claim 16, wherein the oligonucleotide probes are specific for one or more target genomic loci or regions.

18. The method of claim 16, wherein the oligonucleotide probes are specific for known genetic variants.

19. The method of claim 1, further comprising sequencing the target-enriched sequencing library pool thereby generating sequencing reads.

20. The method of claim 19, wherein the sequencing step comprises using a short-read technology.

21. The method of claim 19, wherein the sequencing step comprises using a long-read technology.

22. The method of claim 19, wherein the sequencing step comprises using low-coverage sequencing.

23. The method of claim 22, wherein the low-coverage sequencing comprises providing 10 fold coverage or less of a target genome.

24. The method of claim 19, wherein the sequencing reads are demultiplexed.

25. The method of claim 24, wherein the demultiplexed sequencing reads are aligned to a reference genome.

26-27. (canceled)

28. The method of claim 1, wherein the genetic library is prepared at low-volume.

29. An enriched genetic library comprising a target-enriched subset of a genetic library and an unenriched subset of a genetic library.

30. The enriched genetic library of claim 29, wherein the target-enriched subset and the unenriched subset are separate.

31-34. (canceled)
